(19) United States
(12) Patent Application Publication
(10) Pub. No.: US 2017/0175859 A1
(43) Pub. Date: Jun. 22, 2017

(54) Telescopic Drive Arrangement with Oldham Coupling

(71) Applicant: Novo Nordisk A/S, Bagsvaerd (DK)

(72) Inventor: Pete Brockmeier, Copenhagen V (DK)

(21) Appl. No.: 15/118,123

(22) PCT Filed: Feb. 17, 2015
(86) PCT No.: PCT/EP2015/053296

§ 371 Date: Aug. 11, 2016

(30) Foreign Application Priority Data
Feb. 17, 2014 (EP) 14155378.4

(51) Int. Cl. F16H 25/20; F16H 27/02; A61M 5/315

(52) U.S. Cl. CPC F16H 25/20; A61M 5/315; F16H 27/02; F16D 3/04

(57) ABSTRACT

Telescopic drive assembly comprising a rotatable threaded drive member, an outer tube assembly arranged axially moveable relative to the drive member but non-rotational relative to a frame and comprising a nut portion in threaded engagement with the drive member, a transmitter tube with an outer thread, and a drive tube in threaded engagement with the transmitter tube outer thread. The outer tube assembly comprises a tube guide member coupled to the nut portion via an Oldham coupling.

The present invention generally relates to a telescopic drive arrangement. In a specific aspect the invention relates to such a drive arrangement incorporated in a motorized drug delivery device adapted to receive a drug filled cartridge and subsequently expel a dose therefrom.

### BACKGROUND OF THE INVENTION

In the disclosure of the present invention reference is mostly made to drug delivery devices intended for the treatment of diabetes by subcutaneous drug delivery, either discrete or continuous, however, this is only an exemplary use of the present invention.

The most common type of durable drug delivery devices adapted to receive a drug filled cartridge and expel a discrete dose of a desired size therefrom are driven by manual means or by a spring energized during dose setting, the cartridge being of the type comprising an axially displaceable piston having an initial proximal position and which is moved distally by a piston rod. Subcutaneous drug delivery takes place via an injection needle arranged in fluid communication with the cartridge. The device may be pen-formed or in the form of a more box-shaped so-called doser. In order to improve convenience, user-friendliness and provide additional features, e.g. detection and storing of expelling data, drug delivery devices have been provided with electrically driven means, typically in the form of an electronically controlled motor driving a piston rod through a gear arrangement, e.g. as shown in U.S. Pat. No. 6,514,230 and US 2011/306927.

Although motorized drug delivery devices facilitate a number of benefits to the customer as many trivial routines can be automated, these devices are often rather large and/or long in comparison to conventional mechanical devices and thus they are impractical to carry and store. Addressing this issue WO 2002/034315 discloses a generally pen-formed drug delivery device comprising a drive arrangement including a telescopic piston rod that can be moved into a drug cartridge when being telescopically expanded in a distal direction. The motor for driving the piston rod is arranged co-axially off-set relative to the piston rod to reduce the overall length of the device. Further, WO 97/00091 discloses a telescopic drive arrangement for a medical syringe device.

Although a telescopic piston rod design may result in a compact design, the design involves a number of moving components which interface with multiple other components, all being arranged in a confined space. Such a design will often result in a mechanical design in which the components involved are over-constrained which in turn can lead to malfunction of the device and/or increased production cost and lower production yield.

Having regard to the above, it is an object of the present invention to provide a telescopic drive assembly which ensures a high degree of reliability in a cost-effective way. It is a further object to provide a motorized drug delivery device incorporating such a drive assembly.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in accordance with a first aspect of the invention a drive assembly is provided comprising an elongated drive member, an outer tube assembly, a transmitter tube and a drive tube. The elongated drive member comprises an outer thread defining an axis of rotation, the drive member being arranged axially locked but rotational free relative to the frame. The outer tube assembly is adapted to be arranged axially moveable relative to the drive member but non-rotational relative to the frame, and comprises a nut portion in threaded engagement with the drive member outer thread. The transmitter tube comprises an outer thread and is arranged co-axially with the drive member and inside the outer tube assembly, axially moveable but non-rotational relative to the drive member, and axially locked but rotational free relative to the outer tube assembly. The drive tube is in threaded engagement with the transmitter tube outer thread and is arranged co-axially with the drive member, and axially moveable but non-rotational relative to the outer tube assembly. The outer tube assembly further comprises a tube guide member coupled to the nut portion via an Oldham coupling, e.g. in the form of a coupling ring member.

In accordance with a further aspect of the invention a drive assembly is provided comprising a frame, an elongated drive member, an outer tube assembly, a transmitter tube and a drive tube. The elongated drive member comprises an outer thread defining an axis of rotation, the drive member being arranged axially locked but rotational free relative to the frame. The outer tube assembly is arranged axially moveable relative to the drive member but non-rotational relative to the frame, and comprises a nut portion in threaded engagement with the drive member outer thread. The transmitter tube comprises an outer thread and is arranged co-axially with the drive member and inside the outer tube assembly, axially moveable but non-rotational relative to the drive member, and axially locked but rotational free relative to the outer tube assembly. The drive tube is in threaded engagement with the transmitter tube outer thread and is arranged co-axially with the drive member, and axially moveable but non-rotational relative to the outer tube assembly. The outer tube assembly further comprises a tube guide member coupled to the nut portion via an Oldham coupling, e.g. in the form of a coupling ring member.

By this arrangement the tube guide member and the nut portion is allowed to move relative to each other, the arrangement yet still provides the desired transmission of rotation, this allowing a cost-effective design requiring less restrictive tolerances for the involved components and reduces the need for the components involved being precisely aligned.

In an exemplary embodiment the nut portion comprises a nut member and a base member coupled non-rotationally to each other corresponding to the axis of rotation, the base member being allowed to pivot relative to the nut member corresponding to at least one axis different from the axis of rotation, e.g. perpendicular to the axis of rotation, the nut member being in threaded engagement with the drive member outer thread. The base member is coupled to the tube guide member via the Oldham coupling.

The transmitter tube may be coupled axially locked but rotational free to the base member, and the drive tube may be coupled axially moveable but non-rotational to the tube guide member.

The drive tube may be provided with a number of outer guide projections and the tube guide member may comprises a number of inner guide grooves arranged to receive the guide projections, the guide projections each comprises an outer engagement surface being outwardly curved in the axial and/or circumferential direction, this reducing the need for the components involved to be precisely aligned.

In a further aspect a drug delivery device comprising a drive assembly as described above is provided, the device further comprising a compartment adapted to receive a drug-filled cartridge, the cartridge comprising a body portion, an axially displaceable piston, and a distal outlet portion adapted to be arranged in fluid communication with a flow conduit. The drive tube is adapted to directly or indirectly engage and axially move the piston of a loaded cartridge to thereby expel drug from the cartridge, and electronically controlled drive means is provided to rotate the elongated drive member. The drug delivery device may comprise setting means allowing a user to set a dose of drug to be expelled.

As used herein, the term “drug” is meant to encompass any flowable medicine formulation capable of being passed through a delivery means such as a cannula or hollow needle in a controlled manner, such as a liquid, solution, gel or fine suspension, and containing one or more drug agents. Representative drugs include pharmaceuticals such as peptides (e.g. insulins, insulin containing drugs, GLP-1 containing drugs as well as derivatives thereof), proteins, and hormones, biologically derived or active agents, hormonal and gene based agents, nutritional formulas and other substances in both solid (dispensed) or liquid form. In the description of the exemplary embodiments reference will be made to the use of insulin containing drugs, this including analogues thereof as well as combinations with one or more other drugs.

### BRIEF DESCRIPTION OF THE DRAWINGS

In the following exemplary embodiments of the invention will be further described with reference to the drawings, wherein

FIG. 1A shows in cross-section a conventional telescopic drive assembly,

FIG. 1B shows the drive assembly of FIG. 1A in a partly extended state,

FIG. 2A shows in cross-section a further telescopic drive assembly,

FIG. 2B shows the drive assembly of FIG. 2A in a partly extended state,

FIG. 3 shows an exterior view of the drive assembly of FIG. 2A,

FIG. 4 shows in an exploded view components of the drive assembly shown in FIG. 3,

FIG. 5 shows a motorized drug delivery device, and

FIG. 6 shows in an exploded view components of the drug delivery device shown in FIG. 5

### In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

Before turning to the description of an exemplary embodiment of the invention a conventional telescopic drive assembly will be described to better provide an understanding of the general working principle of such an arrangement.

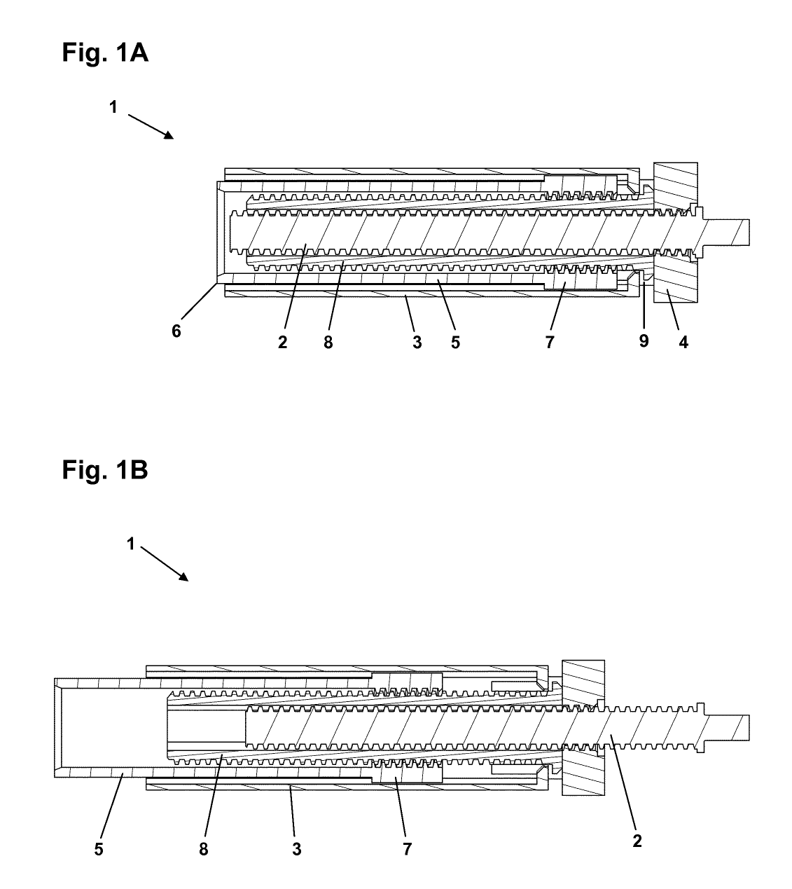

More specifically, FIG. 1A shows a telescopic drive assembly 1 comprising a threaded lead screw 2, an outer tube 3 with a threaded proximal portion 4, an inner drive tube 5 with a distal free end 6 and a proximal threaded portion 7, and an externally threaded transmitting tube 8. In an operational state the assembly is mounted in a frame (not shown). The lead screw defines an axial direction as well as a rotational z-axis.

The lead screw 2 is rotationally driven at its proximal end about the z-axis by some means and interfaces with three components: a z-axis translation lock with the frame (not shown), a thread interface with the outer tube proximal threaded portion 4, and a sliding rotation lock interface with the transmitting tube 8. As the lead screw rotates, the outer tube, which has a sliding rotation lock interface to the frame, translates along the z-axis at a speed determined by the pitch and the rotational speed of the lead screw. The transmitting tube 8 has three interfaces: a sliding rotation lock interface with the lead screw, a z-axis translation lock 9 with the outer tube, and a thread interface with the drive tube proximal threaded portion 7. As the lead screw rotates, the sliding rotation lock forces the transmitting tube to rotate at the same speed. As the transmitting tube rotates, the inner drive tube, which has a sliding rotation lock interface to the outer tube, translates along the z-axis at a speed determined by the pitch and the rotational speed, plus the translational speed of the outer tube. As appears, when the lead screw is rotated the outer tube is moved distally and the inner tube is “telescoped” distally out of the outer tube, this as shown in FIG. 1B.

For proper function of a telescopic drive assembly all interfaces must remain free to translate and/or rotate along relatively long distances as the inner tube is driven forward for which reason small tolerances on components may be required to ensure that the components maintain movability. Additionally, it may be desirable to keep the dimensions of the telescopic drive assembly as small as possible by minimizing clearance between components. This can require additionally small component tolerances. Addressing these issues embodiments of the present invention provide special kinematic joints to allow additional degrees of freedom between components.

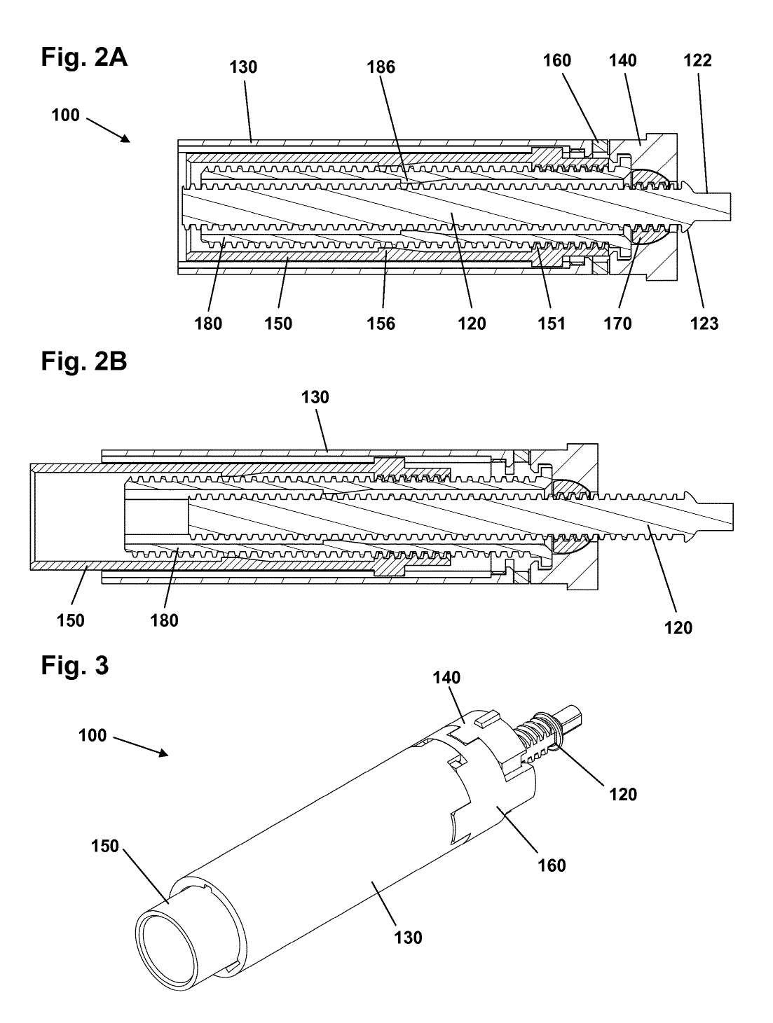

Turning to an exemplary embodiment of the invention FIG. 2A discloses a telescopic drive assembly 100 comprising a threaded lead screw 120, an outer tube assembly, an inner drive tube 150, and a transmitting tube 180. The components essentially have the same functional relationship as in the above-described conventional design, however, the outer tube assembly replacing the outer tube member comprises in the shown embodiment four members in the form of a guide tube 130 coupled to a base member 140 via a generally ring-formed Oldham coupling member 160, as well as a nut member 170 housed in the base member. FIG. 2B shows the telescopic drive assembly 100 in a partly telescoped state corresponding to the state shown in FIG. 1B.

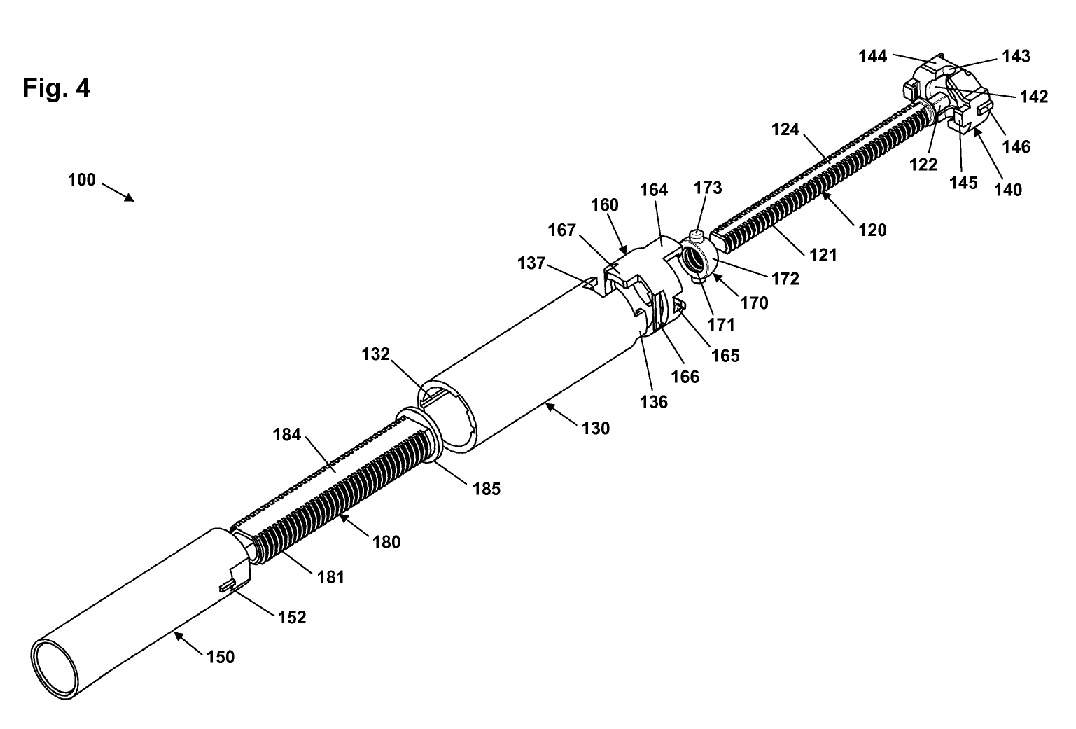

In the following with reference to FIGS. 2A, 3 and 4 a more detailed description of the individual components of the telescopic drive assembly 100 will be given. More specifically, the lead screw 120 comprises an outer thread 121, a proximal end 122 with a pair of opposed flat surfaces serving as an input shaft and allowing a drive member, e.g. a gear wheel, to be mounted, as well as a part-spherical surface portion 123 adapted to engage a corresponding seat in a frame member. Along the length of the thread two opposed co-planar sliding surfaces 124 are provided adapted to engage corresponding inner surfaces on the transmitting tube 180 to thereby provide a sliding rotation lock interface there between.

The nut member 170 comprises a part-spherical surface portion 172 adapted to engage a corresponding seat 142 in the base member, a pair of opposed radially projecting round hinge pins 173 adapted to engage corresponding hinge seats 143 in the base member, as well as an internal thread adapted to engage the lead screw outer thread 121, the pins providing a rotational lock in the z-axis yet allows the nut member to pivot (typically a few degrees of rotation) corresponding to the hinge axis defined by the pins. Alternatively a further hinge could be provided allowing the nut member to move “cardan-wise” in the base member.

The base member 140 further comprises a pair of opposed co-planar sliding surfaces 144 adapted to engage corresponding surfaces on a pair of proximally projecting hook portions 164 on the Oldham coupling member thereby provide a sliding rotation lock interface there between, a pair of opposed distally projecting hook portions 145 adapted to grip and engage a proximal flange portion 185 on the transmitting tube to thereby provide a z-axis translation lock, as well as a pair of axially extending opposed guide projections 146 adapted to engage corresponding guide slots formed in a frame member to thereby provide a spline sliding rotation lock interface there between. The outer surfaces of the guide projections may be curved in the axial and/or circumferential direction in order to reduce the need for the components involved to be precisely aligned. The opposed hook portions 145 are off-set 90 degrees relative to the opposed sliding surfaces 144.

The Oldham coupling member 160 comprises the above-described hook portions 164 which comprise inwards directed hook edges adapted to axially lock the coupling member to the base member, as well as a pair of opposed transverse outer ridge structures 166 arranged 90 degrees off-set relative to the hook portions 164 and adapted to engage corresponding surfaces on a pair of proximally projecting hook portions 136 on the guide tube thereby provide a sliding rotation lock interface there between. To further provide rotational lock the Oldham coupling member comprises a pair of opposed cut-outs 165 adapted to receive the base member hook portions 145, as well as a pair of opposed distally directed extensions 167 of the hook portions 164, the extensions being adapted to be received in a pair of corresponding cut-outs 137 in the guide tube.

The guide tube 130 comprises the above-described hook portions 136 and cut-outs 165 as well as a pair of axially extending opposed guide grooves 132 adapted to engage corresponding proximal guide projections 152 formed on the inner drive tube 150 to thereby provide a spline sliding rotation lock interface there between. The outer surfaces of the guide projections may be curved in the axial and/or circumferential direction in order to reduce the need for the components involved to be precisely aligned.

The transmitting tube 180 comprises an outer thread 181 adapted to engage a corresponding proximal inner thread 151 on the inner drive tube (see FIG. 2A), a pair of opposed co-planar wall portions 184 providing the above-described inner surfaces engaging the lead screw sliding surfaces, the above-described proximal flange portion 185 held axially locked by the base member hook portions 145, as well as a pair of opposed inner axially oriented projections 186 adapted to support the lead screw (see FIG. 2A).

The inner drive tube 150 comprises the above-described inner thread 151 and guide projections 152, as well as a pair of opposed inner axially oriented projections 156 adapted to support the transmitting tube.

As appears, the described telescopic drive assembly 100 comprises two distinct arrangements, each providing increased flexibility of the drive assembly without hampering the transmission efficiency of the assembly. More specifically, the combined interfaces of the Oldham coupling member 160 with the base member 140 respectively the guide tube 130 provide that the guide tube can move in a plane perpendicularly to the rotational z-axis of the base member, yet provide a rotational lock between the base member and the guide tube. Correspondingly, the hinge formed between the nut member and the base member allows the nut member and thereby the lead screw to pivot corresponding to the hinge axis, yet provides a rotational lock between the nut member and the base member. As the two arrangements are distinct and independent, in a first alternative embodiment the base member and guide member may be formed as a single element, just as in a second alternative embodiment the base member, the Oldham coupling member and the guide tube may be formed as a single element.

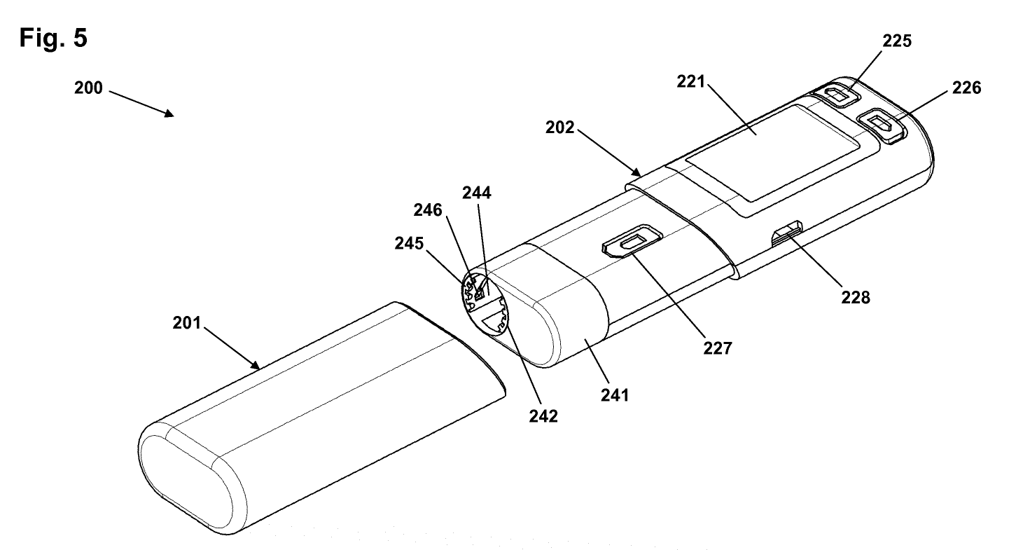

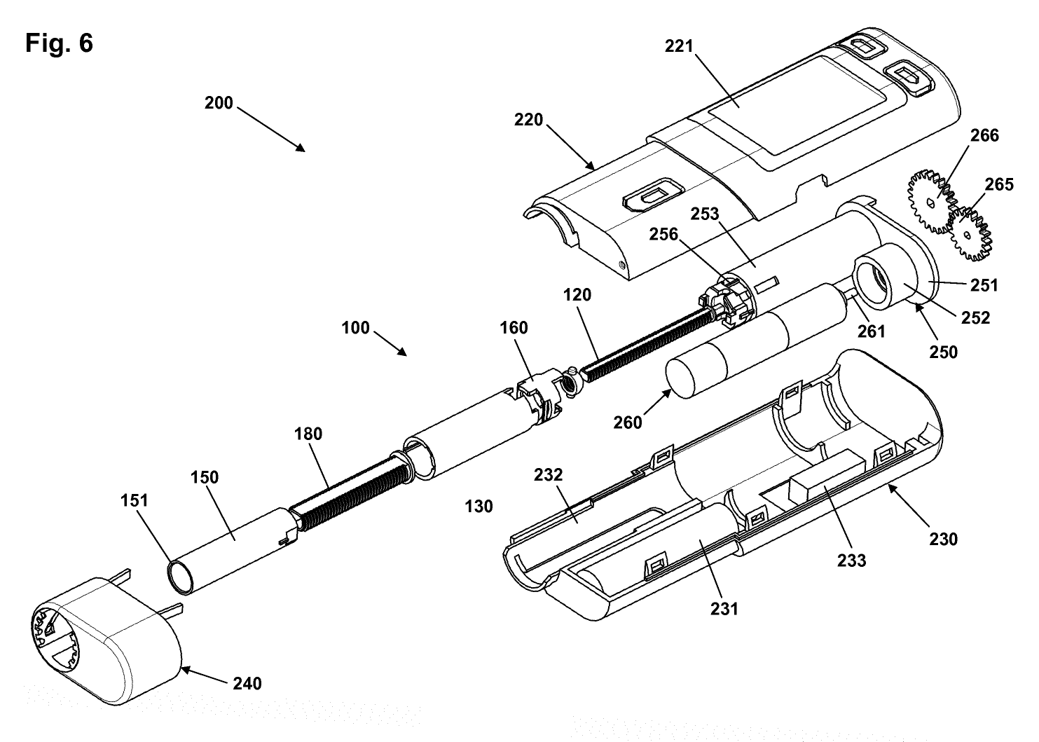

Referring to FIGS. 5 and 6 a motorized “box-formed” drug delivery device 200 will be described, the device being adapted to receive a drug cartridge comprising a cylindrical body portion, a distal outlet portion with a distal needle-penetrable septum, a proximal open-ended portion, and an axially displaceable piston having a proximal surface allowing a piston driver forming part of the expelling mechanism (see below) to engage the piston. The cartridge may for example contain a drug in the form of an insulin, a GLP-1 or a growth hormone formulation. The cartridge may be provided with distal coupling means in the form of a needle hub mount.

More specifically, the pen device comprises a cap part 201 and a main part 202 formed by an upper housing member 220 and a lower housing member 230, the housing members forming an interior having a proximal drive assembly portion in which a drug expelling mechanism and associated controller electronics 233 are arranged, and a distal portion in which a rechargeable energy source 231 (battery) is arranged. The distal portion further comprises a front-loaded cartridge holder compartment 232 adapted to receive a drug-filled transparent cartridge through a distal opening 242, a received cartridge being retained in place by a cartridge holder locking assembly 240 mounted to the housing members. A spring-actuated axially moveable cartridge bias member (not shown) is arranged proximally in the cartridge holder compartment. A corresponding cartridge bias member is described in EP application 14159913.4.

The upper housing member comprises a window 221 through which a display (not shown) can be observed by a user, as well as user input keys. In the shown embodiment a pair of dose setting input keys 225, 226 serves to manually set a desired dose of drug shown in the display and which can then be expelled when a distally arranged release button 227 is actuated. A side opening 228 allows an electrical connector, e.g. USB, to be inserted, the connector providing charging of the battery as well as allowing data transmission to and/or from an external device, e.g. a PC. Additionally, the controller electronics may be associated with or comprise a receiver and/or transmitter allowing the device to communicate with an external source by wireless means such as e.g. Bluetooth, NFC or Wi-Fi. In this way a log of expelled doses could be transferred to a PC or smartphone or the smartphone could be used to conveniently enter pre-set dose sizes.

The drug expelling mechanism comprises a frame member 250, a motor and gear assembly 260, and a telescopic drive assembly 100 as described above, the inner drive tube 150 comprising a distal portion 151 adapted to engage the proximal free surface of the piston of a loaded cartridge. Not forming part of the present invention, the drive tube distal portion may be provided with electronic sensor means for detection of contact between the drive tube and the piston, e.g. using proximity detection as disclosed in WO 2013/144152. Alternatively, contact could be detected by monitoring the load on the motor, e.g. by monitoring the current. The frame member 250 comprises a base plate portion 251, a short first tubular support 252 adapted to receive the motor and gear assembly, and a longer co-axially arranged second tubular support 253 adapted to receive the telescopic drive assembly. When mounted the rotating output shaft 261 of the motor and gear assembly projects through a first opening in the base plate portion, and the input shaft of the telescopic drive assembly projects through a second opening in the base plate, the two shafts being rotationally coupled by a pair of transmission gear wheels 265, 266 mounted on the output respectively the input shafts. The second tubular support comprises a pair of opposed guide grooves 256 adapted to engage the base member guide projections 146 thereby providing a sliding rotation lock interface between the frame and the base member and thereby also the guide tube.

The cartridge holder locking assembly 240 comprises a housing 241 with a receiving opening 242 and a pair of opposed flexible cartridge holder arms 244, each arm being provided with a gripping shoulder 245. The housing serving as an actuation member is mounted on the device main part and adapted to rotate e.g. 90 degrees corresponding to an axis through the centre of the receiving opening, rotation of the actuation member back and forth controlling movement of the gripping shoulders in and out to a closed respectively open position. In the shown embodiment the gripping shoulders 245 is provided with a plurality of gripping teeth spaced circumferentially to provide a plurality of gaps intended to grip a cartridge hub mount as provided on a Penfill® manufactured and sold by Novo Nordisk A/S, Denmark, and described in U.S. Pat. No. 5,693,027. The actuation member has an operational position in which it is rotated to be flush with the main housing and the cartridge holder arms retracted to its operational closed holding position, and a loading/un-loading position in which the actuation member is rotated to be non-flush with the main housing and the cartridge holder arms are extended to an open loading position allowing a cartridge to be removed and replaced with a new. In the shown embodiment movement of the cartridge holder arms relative to the actuation member is controlled by housing guide projections received in corresponding arm guide tracks 246. The gripping shoulders may be adapted to grip a partially inserted cartridge and move it proximally to a fully inserted position against the force of the biasing means. A front-loaded cartridge holder assembly of the above-described type is disclosed in WO 2013/124119.

When a new cartridge is inserted the drive expelling means has to be in a state allowing a new cartridge with a proximally positioned piston to be inserted, i.e. the drive tube 150 has to be retracted. This may be done either manually by the user operating input keys to have the motor retract the drive tube, or automatically by switch means operated by the locking assembly or an inserted cartridge. For example, a switch may be arranged to be operated by the bias member, the switch having an initial first state when the bias member is in an initial un-loaded distal position and an actuated second state when the bias member is moved proximally to an actuated position. The bias member is adapted to engage a proximal portion of an inserted cartridge, thereby exerting a distally directed biasing force, and to move a loaded cartridge at least partly through the receiving opening when the cartridge holder locking assembly is actuated from the closed state to the open state. In such an arrangement a retracted drive tube 150 is moved distally to engage the piston of a loaded cartridge when the switch means is actuated from the first state to the second state as a cartridge is loaded and locked in place, and the drive tube is retracted when the switch means is actuated from the second state to the first state when a loaded cartridge is released and moved distally. A corresponding switch arrangement is described in EP application 14159913.4.

When the user desires to remove a cartridge (which may be fully or partly empty), the user rotates the actuation member to its loading/un-loading position whereby the cartridge holder gripping shoulders are moved to their open position, this allowing the cartridge to be pushed proximally by the distally directed biasing force of the bias member. To prevent that the cartridge “shoots out” or slides out of the cartridge holder, a slight friction may be provided between the cartridge holder and a loaded cartridge. As the bias member is moved distally by the spring the bias member disengages the cartridge switch. The actuation of the cartridge switch from closed to open signals to the device controller that two actions can be assumed to have taken place: (i) the cartridge holder has been opened and (ii) an inserted cartridge has been pushed distally by the bias member, this initiating retraction of the drive tube to its fully retracted position corresponding to FIG. 2A. As it will take some time to retract the piston tube the user will normally remove the cartridge before the piston tube has been fully retracted, however, as the piston tube in all positions are positioned fully inside the cartridge holder it is protected against unintended damage. During normal use situations it can be assumed that the piston tube is fully retracted when the user inserts a new cartridge, however, to prevent a “too fast” insertion of a new cartridge the user may be instructed to await a signal indicating that the device is ready to receive a cartridge, e.g. a sound signal.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drive assembly comprising:
a frame,
an elongated drive member comprising an outer thread and defining an axis of rotation, the drive member being arranged axially locked but rotational free relative to the frame,
an outer tube assembly arranged axially moveable relative to the drive member but non-rotational relative to the frame, comprising a nut portion in threaded engagement with the drive member outer thread,
a transmitter tube comprising an outer thread and being arranged:
co-axially with the drive member and inside the outer tube assembly,
axially moveable but non-rotational relative to the drive member, and
axially locked but rotational free relative to the outer tube assembly,
a drive tube in threaded engagement with the transmitter tube outer thread and being arranged:
co-axially with the drive member, and
axially moveable but non-rotational relative to the outer tube assembly,
wherein the outer tube assembly further comprises a tube guide member coupled to the nut portion via an Oldham coupling.

2. A drive assembly as in claim 1, wherein:
the nut portion comprises a nut member and a base member coupled non-rotationally to each other corresponding to the axis of rotation, the base member being allowed to pivot relative to the nut member corresponding to at least one axis perpendicular to the axis of rotation,
the nut member is in threaded engagement with the drive member outer thread, and
the base member is coupled to the tube guide member via the Oldham coupling.

3. A drive assembly as in claim 2, wherein:
the transmitter tube is coupled axially locked but rotational free to the base member.

4. A drive assembly as in claim 1, wherein:
the drive tube is coupled axially moveable but non-rotational to the tube guide member.

5. A drive assembly as in claim 4, wherein:
the drive tube comprises a number of outer guide projections and the tube guide member comprises a number of inner guide grooves arranged to receive the guide projections, and
the guide projections each comprises an outer engagement surface being outwardly curved in the axial direction.

6. A drive assembly as in claim 1, wherein the Oldham coupling is in the form of a coupling ring member arranged between the tube guide member and the nut portion.

7. A drug delivery device comprising a drive assembly as in claim 1, further comprising:
a compartment adapted to receive a drug-filled cartridge, the cartridge comprising a body portion, an axially displaceable piston, and a distal outlet portion adapted to be arranged in fluid communication with a flow conduit,
wherein the drive tube is adapted to directly or indirectly engage and axially move the piston of a loaded cartridge to thereby expel drug from the cartridge, and
electronically controlled drive structure adapted to rotate the elongated drive member.

8. Drug delivery device as in claim 7, further comprising setting structure allowing a user to set a dose of drug to be expelled.

9. Drug delivery device as in claim 7, wherein the compartment comprises a distal opening allowing a drug-filled cartridge to be received in a proximal direction.
